(19) United States
(12) Patent Application Publication
(10) Pub. No.: US 2022/0127758 A1
(43) Pub. Date: Apr. 28, 2022

(54) METHOD OF FORMING ELASTIC FIBERS AND STRETCHABLE ARTICLES CONTAINING SUCH FIBERS

(71) Applicant: DOW GLOBAL TECHNOLOGIES LLC, Midland, MI (US)

(72) Inventors: Aleksandar STOILJKOVIC, Horgen (CH); Barbara BONAVOGLIA, Horgen (CH); Jill M. MARTIN, Freeport, TX (US)

(21) Appl. No.: 17/427,755

(22) PCT Filed: Feb. 11, 2020
(86) PCT No.: PCT/US2020/017614

§ 371 Date: Aug. 2, 2021

Related U.S. Application Data
(60) Provisional application No. 62/806,159, filed on Feb. 15, 2019.

(51) Int. Cl. D01F 6/30; D01D 5/088; D01D 5/16; A61F 13/49

(52) U.S. Cl. CPC D01F 6/30; D01D 5/088; D10B 2401/061; A61F 13/4902; D10B 2321/02; D01D 5/16

(57) ABSTRACT

A method comprises providing a resin comprising an olefin block copolymer; spinning the resin into one or more fibers; cooling the one or more fibers to below the solidification point of the resin; after cooling, stretching the one or more fibers to a nominal elongation of 50 to 900% to form one or more stretched fibers; and relaxing the one or more stretch fibers to form one or more elastic fibers, and, optionally laminating the fibers to a flexible substrate. Articles comprising such elastic fiber(s) bonded to a nonwoven, can exhibit: a force at 50% elongation of less than 2 Newtons, a force at 100% elongation of less than 4 Newtons, a elongation at five Newtons for a 50 mm sample of at least 120%, and/or an unload force at 50% on a second cycle of less than 1.5 N/50 mm.

### CROSS REFERENCE TO RELATED APPLICATIONS

This application claims the benefit of U.S. application Ser. No. 62/806,159, filed on Feb. 15, 2019, which is incorporated herein by reference in its entirety.

### FIELD OF THE INVENTION

The field of this invention is a method of forming elastic fibers and stretchable articles containing such fibers.

### BACKGROUND

In products such as diapers and incontinence products, elastic side panels or elastic flaps (sometimes called elastic ears) or the like are often used. Some of these elastic components have been made from non-woven layers laminated to elastic sheets or filaments. However, it can be challenging to efficiently manufacture a product that provides comfort, versatility, and overall quality.

### SUMMARY OF THE INVENTION

Disclosed herein is a method of making an elastic article comprising providing a resin having a peak melting temperature as measured by differential scanning calorimetry (DSC) and comprising an olefin block copolymer; spinning the resin into one or more fibers; cooling the one or more fibers to below the solidification point of the resin; after cooling, stretching the one or more fibers to a nominal elongation of 50 to 900% to form one or more stretched fibers; and relaxing the one or more stretch fibers to form one or more elastic fibers.

The method can further comprise laminating the one or more stretched fibers to a flexible substrate.

Also disclosed herein are elastic fibers and elastic articles made according to such a method. Particularly, disclosed herein is an elastic article comprising one or more elastic fibers bonded to a nonwoven, wherein the one or more elastic fibers comprises olefin block copolymer and wherein the laminate exhibits one or more of the following properties: a force at 50% elongation of less than 2 Newtons (N), a force at 100% elongation of less than 4 Newtons, an elongation at five Newtons for a 50 millimeter (mm) sample of the laminate of at least 120%, an unload force at 50% on a second cycle of less than 1.5 N/50 mm.

### BRIEF DESCRIPTION OF THE DRAWINGS

FIG. 1 is a schematic illustrating an apparatus for performing an embodiment of the method disclosed herein.

### DETAILED DESCRIPTION OF THE INVENTION

The method of making the article and the articles made according to the method can show a good balance of properties including one or more of the following: the elastic fibers show relatively low hysteresis loss demonstrating good elastic performance; the elastic fibers and/or elastic articles show relative low force at elongation indicating a gentle stretch which leads to more comfort in wearing a product made with the article; the elastic fibers and/or elastic articles show relatively high elongation which enables a manufacturer of diapers or incontinence products to reduce the number of sizes it produces and sells.

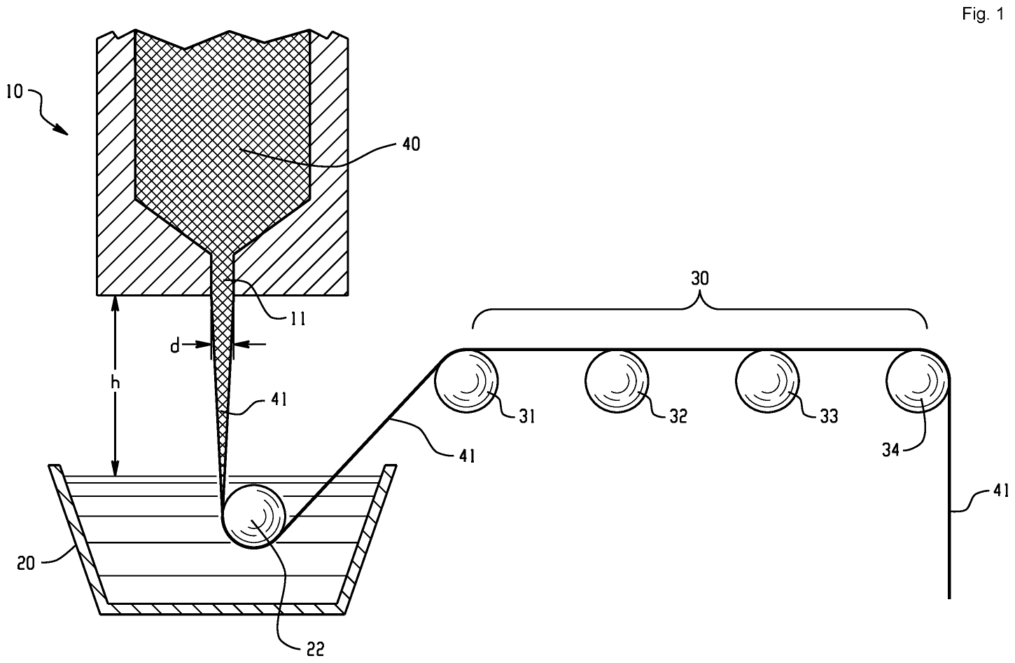

Referring to FIG. 1, the method disclosed herein can include providing an extruder and die system 10, where resin 40 is heated to a molten state and extruded through an orifice or hole 11 to form a fiber 41 which is drawn down using a roller 22 to a desired cross section dimension and cooled in a water bath 20. As shown, only one extruder and die system 10, one hole 11 and one fiber 41 is being shown. However, it is contemplated that multiple fibers may be formed from multiple holes in one die, and/or from more than one die and/or more than one extruder. In the water bath, the fiber is cooled to a solidification point. After cooling, fiber 41 is stretched in a stretching zone 30. While any known stretching mechanism can be used, according to the example shown in FIG. 1 the stretching zone 30 can comprise a series of two or more (four are shown rollers) 31, 32, 33, 34. A stretch ratio can be defined as the speed or velocity of the final roller (e.g. roller 34 in FIG. 1) over the speed or velocity of the first roller (e.g. roller 31 in FIG. 1). The fiber 41 (or fibers if multiple are being processed) can then be collected. For example, the fiber(s) can be collected by an air gun into a bag and/or wound onto a roll. The winding can be done without additional stretching or tension on the fibers. The size of the hole 11 is designated by the dimension, d. The distance from the die to the surface of the water bath is designated by dimension, h.

The extruder can be any known extruder type useful in making fibers or filaments. The extruder can heat the resin to its molten state and push the resin through the hole or holes in the die. The extruder for example may heat the resin to temperatures in the range of at least 100 or at least 150 ° C. up to 250° C.

The die contains one or more holes or orifices 11 through which the molten resin 40 is extruded. The shape of the holes may be any regular or irregular shape as desired (e.g. circular, elliptical, polygonal, hexagonal, octagonal, triangular, square, etc.). The maximum cross section, d, of the hole(s) (e.g. diameter in the case of a circular opening) is according to certain embodiments at least 0.2 or at least 0.4 mm up to 6, up to 5, up to 4, or up to 3 mm.

The draw down decreases the cross section of the fiber relative to the cross section of the hole.

After draw down, the fiber is cooled to a temperature at or below the resin's solidification point. As used herein, solidification point means the temperature at which the viscosity of a resin reaches 40,000 Pascal-seconds (Pa·s), and is reported in degrees Celsius (° C.). FIG. 1 shows a water bath 20 that may be used for cooling the fiber 41 to or past the solidification point. Other means for cooling, such as cool air, could be used in place of a water bath. For example, the cooling medium can have a heat transfer coefficient of at least 100 or at least 1000 or at least 2000 W/m²*K (watts per square meter per degree Kelvin). The heat transfer coefficient can be less than 10,000 W/m²*K. A water bath or cooling medium with a similar or better heat transfer coefficient is particularly suitable for thicker fibers. When a water bath is used as shown in FIG. 1, the water temperature can be kept in the range of from 10, from 15, or from 20° C. to 40 or to 30° C. The fiber(s) is (are) kept in the water for a time sufficient to ensure that it has passed its solidification point. For example, the water path (i.e., the distance that the fiber is in the water) can be from 10 or from 20 to 100, to 80, or to 60 centimeter (cm). The distance, h, is an air gap and can be selected to enable adequate draw down before quench. For example, h can be from 5 or from 10 cm to 50, to 40, to 30, or to 20 cm.

After cooling (e.g. quench) the method disclosed herein includes stretching the one or more fibers to a nominal elongation in the range of 50% to 900%. Any means known in the art for stretching may be used. For example, a series of two or more rollers (can be used for stretching where the fibers are moved by and over the rollers. Four rollers 31, 32, 33, and 34 are shown in FIG. 1. The first roller, e.g. roller 31, is at the lowest speed and the final roller, e.g. roller 34, is at the highest speed. Intermediate rollers, e.g. rollers 32 and 33, if any, are at least as fast as the prior roller and no faster than the subsequent roller. Each roller in the series can be at a higher speed than the preceding roller. Use of rollers for stretching can be particularly useful for continuous long lengths of fiber. Alternatively, for example, fibers may be anchored at one end and pulled at another end to a desired extension. This anchored method of stretching can be useful for stretching cut fibers of a more limited length.

The fibers can be stretched to a nominal elongation of at least 50, at least 100, at least 150, or at least 200%. The fibers can be stretched to a nominal elongation of no more than 900, no more than 800, no more than 700, no more than 600, no more than 500 or no more than 400%. Nominal elongation refers to the elongation that would be achieved by the stretching if there is no slippage or other inefficiency in the stretching. In a stretching mechanism where the fiber is anchored and pulled the nominal elongation in percent will be 100×[(the length of the fiber when fully extended)−(the original length of the fiber)]/(the original length of the fiber). In a stretching mechanism where the fiber is stretched over rollers, the nominal elongation in percent will be 100×[(speed of final roller)−(speed of first roller)]/(speed of the first roller). In this mechanism, there may be some slippage of the fiber on the rolls (although desirably that is avoided or minimized), such that actual elongation may be slightly less than the nominal elongation.

When stretching over rollers, the amount of stretch can alternatively be defined as a stretch ratio. The stretch ratio is defined as the speed or velocity of the final roller (e.g. roller 34) divided by the speed or velocity of the first roller (e.g. roller 31). The stretch ratio can be at least 1.5 or at least 2. The stretch ratio can be no more than 10, no more than 8, or no more than 6. The rollers used for stretching are driven at the desired speeds to achieve the desired stretch ratio. For example, the first roller may have a velocity of from 10 or from 50 or from 70 or from 100 meters/minute (m/min) up to 300, up to 250, up to 200, or up to 100 meters/minute (m/min) and the final roller may have velocity of more than 300 m/min up to 400 m/min or more than 400 m/min up to a desirable rate such as for example 500 or 600 m/min.

According to certain embodiments, the rollers for stretching may be heated slightly. Such elevated temperature facilitates good contact and friction between the fibers and the rollers, which assists with stretching and avoiding the fibers sliding on the rollers. If the temperature of the rollers is too low, there can be a significant amount of sliding on the rollers which may lead to less stretching of the fibers. If the temperature is too high, fibers may stick to the rollers or break. For example, the temperature of the rollers can be at least 35, at least 40, at least 45, at least 50, or at least 60° C. The temperature of the rollers can be no more than 100, no more than 90, or no more than 80° C. For example, the temperature of the rollers can be in the range of 60 to 80° C.

The method further comprises relaxing the one or more fibers by releasing the tension from the fiber(s) after the stretching. The relaxing may occur before or after the step of laminating the one or more fibers to one or more flexible substrates, as discussed below. The relaxing can occur within less than 5, less than 4, less than 3, less than 2, or less than 1 minute or less than 45, less than 30, less than 15, less than 10 or less than 5 seconds after stretching is complete.

The fibers can be collected for future use. For example, they may be collected in a bag or on a roll. Desirably, if collected on a roller that roller is at the same speed or less than the speed of the final stretching roller so as not to be storing the fiber on the roller at tension.

The method can further comprise laminating the fibers to a flexible substrate. For example, while still under tension the fiber(s) can be immediately brought into contact with and laminated to a flexible substrate. In this instance, the relaxing step occurs after lamination. Alternatively, the fibers can be relaxed and then later laminated to a flexible substrate. In certain examples the fibers may be restretched and elongated when laminated to the flexible substrate and relaxed again after lamination. The fibers can be laminated between two flexible substrates (e.g. non-woven materials).

The flexible substrate can be a plastic film or a woven or non-woven fabric. The flexible substrate can be a non-woven fabric as are known to be used in diapers and/or incontinence products.

Laminating may occur by any method known in the art. Adhesive may be used to facilitate lamination and improve adhesion of the fiber(s) to the flexible substrate if needed or desired. The adhesive may be continuous or discontinuous. Examples of suitable adhesives include glues, hot melt adhesives, and the like.

The fiber(s) can be extended to an elongation of at least 50, at least 150, at least 200, or at least 250% before and/or during lamination. The fiber(s) can be extended to an elongation of no more than 400 or no more 350% before and/or during lamination.

The laminate as disclosed herein can be characterized by a force at 50% elongation of up to 2.3, up to 2, or up to 1.8 Newtons for a 50 mm wide sample. The laminate can be characterized by a force at 50% elongation of at least 0.5 or at least 1 Newtons for a 50 mm wide sample. The laminate can be characterized by a force at 100% elongation of up to 4.5, up to 4, up to 3.5., or up to 3 Newtons for a 50 mm wide sample. The laminate can be characterized by a force at 100% elongation of at least 1 or at least 1.5 Newtons for a 50 mm wide sample. The laminate can be characterized by an elongation at a force of 5 N for a 50 mm sample of at least 100, at least 120, at least 140, or at least 150%. The laminate can be characterized by an elongation at a force of 5 N for a 50 mm sample of no more than 250 or no more than 200%. The laminate can be characterized by an elongation at maximum force in the range of at least 250, at least 300, or at least 350 up to 450 or up to 420%. The laminate can be characterized by a maximum force of at least 30 or at least 50 N/50 mm sample. A maximum force for a 50 mm samples can be up to 70 or up to 65 Newtons. The laminate can be characterized by an unload force as described herein at 50% for 2 cycles of up to 1.5, up to 1.2, or up to 1.0 and at least 0.3, at least 0.4 or at least 0.5 Newtons for a 50 mm wide sample. The tests are run according to ISO527-3 as described herein on 50 mm wide samples.

The method utilizes olefin block copolymers in formation of the fibers. The term “olefin block copolymer” or “OBC” means (and is interchangeable with) an “ethylene/α-olefin multi-block interpolymer” or an “ethylene/α-olefin multi-block copolymer” and includes ethylene and one or more copolymerizable α-olefin comonomer in polymerized form, characterized by multiple blocks or segments of two or more polymerized monomer units differing in chemical or physical properties. The terms “interpolymer” and “copolymer” are used interchangeably herein. When referring to amounts of “ethylene” or “comonomer” in the interpolymer, it is understood that this means polymerized units thereof. In some embodiments, the olefin block copolymer is an ethylene/α-olefin multi-block interpolymer. In some embodiments, the ethylene/α-olefin multi-block copolymer can be represented by the following formula:

(AB)ₙ,

where n is at least 1, for example n can be 1 or an integer greater than 1, such as 2, 3, 4, 5, 10, 15, 20, 30, 40, 50, 60, 70, 80, 90, 100, or higher, “A” represents a hard block or segment and “B” represents a soft block or segment. “A”s and “B”s can be linked, or covalently bonded, in a substantially linear fashion, or in a linear manner, as opposed to a substantially branched or substantially star-shaped fashion. A blocks and B blocks can be randomly distributed along the polymer chain. In other words, the block copolymers usually do not have a structure as follows.

AAA-AA-BBB-BB.

The block copolymers can be free of a third type of block, which comprises different comonomer(s). Each of block A and block B can have monomers or comonomers substantially randomly distributed within the block. In other words, neither block A nor block B comprises two or more sub-segments (or sub-blocks) of distinct composition, such as a tip segment, which has a substantially different composition than the rest of the block.

Ethylene can comprise the majority mole fraction of the whole block copolymer, i.e., ethylene comprises at least 50 mole percent of the whole polymer. For example, ethylene can comprise at least 60 mole percent, at least 70 mole percent, or at least 80 mole percent, with the substantial remainder of the whole polymer comprising at least one other comonomer that is preferably an α-olefin having 3 or more carbon atoms, or 4 or more carbon atoms. Octene is an example of such alpha-olefin. The ethylene/α-olefin multi-block copolymer can comprise at least 50, at least 60, or at least 65 mol % to 90, to 85, or to 80 mol % ethylene. For many ethylene/octene multi-block copolymers, the composition comprises an ethylene content greater than 80 mole percent of the whole polymer and an octene content of from 10 to 15, or from 15 to 20 mole percent of the whole polymer.

The ethylene/α-olefin multi-block copolymer can include various amounts of “hard” segments and “soft” segments. “Hard” segments are blocks of polymerized units in which ethylene is present in an amount greater than 90 weight percent, or 95 weight percent, or greater than 95 weight percent, or greater than 98 weight percent based on the weight of the polymer, up to 100 weight percent. In other words, the comonomer content (content of monomers other than ethylene) in the hard segments is less than 10 weight percent, or 5 weight percent, or less than 5 weight percent, or less than 2 weight percent based on the weight of the polymer, and can be as low as zero. In The hard segments can include all, or substantially all, units derived from ethylene. “Soft” segments are blocks of polymerized units in which the comonomer content (content of monomers other than ethylene, such as alpha olefins having 3 or more or 4 or more carbon atoms) is greater than 5 weight percent, or greater than 8 weight percent, greater than 10 weight percent, or greater than 15 weight percent based on the weight of the polymer. The comonomer content in the soft segments can be greater than 20 weight percent, greater than 25 weight percent, greater than 30 weight percent, greater than 35 weight percent, greater than 40 weight percent, greater than 45 weight percent, greater than 50 weight percent, or greater than 60 weight percent and can be up to 100 weight percent.

The soft segments can be present in an ethylene/α-olefin multi-block copolymer from 1 weight percent to 99 weight percent of the total weight of the ethylene/α-olefin multi-block copolymer, or from 5 weight percent to 95 weight percent, from 10 weight percent to 90 weight percent, from 15 weight percent to 85 weight percent, from 20 weight percent to 80 weight percent, from 25 weight percent to 75 weight percent, from 30 weight percent to 70 weight percent, from 35 weight percent to 65 weight percent, from 40 weight percent to 60 weight percent, or from 45 weight percent to 55 weight percent of the total weight of the ethylene/α-olefin multi-block copolymer. Conversely, the hard segments can be present in similar ranges. The soft segment weight percentage and the hard segment weight percentage can be calculated based on data obtained from DSC or NMR (nuclear magnetic resonance). Such methods and calculations are disclosed in, for example, U.S. Pat. No. 7,608,668, entitled “Ethylene/α-Olefin Block Interpolymers,” filed on Mar. 15, 2006, in the name of Colin L. P. Shan, Lonnie Hazlitt, et al. and assigned to Dow Global Technologies Inc., the disclosure of which is incorporated by reference herein in its entirety. In particular, hard segment and soft segment weight percentages and comonomer content can be determined as described in Column 57 to Column 63 of U.S. Pat. No. 7,608,668.

The ethylene/α-olefin multi-block copolymer is a polymer comprising two or more chemically distinct regions or segments (referred to as “blocks”) preferably joined (or covalently bonded) in a linear manner, that is, a polymer comprising chemically differentiated units which are joined end-to-end with respect to polymerized ethylenic functionality, rather than in pendent or grafted fashion. The blocks can differ in one or more of the following: the amount or type of incorporated comonomer, density, amount of crystallinity, crystallite size attributable to a polymer of such composition, type or degree of tacticity (isotactic or syndiotactic), regio-regularity or regio-irregularity, amount of branching (including long chain branching or hyper-branching), homogeneity or any other chemical or physical property. Compared to block interpolymers of the prior art, including interpolymers produced by sequential monomer addition, fluxional catalysts, or anionic polymerization techniques, the present ethylene/α-olefin multi-block copolymer can be characterized by unique distributions of both polymer polydispersity (PDI or Mw/Mn or molecular weight distributin (MWD) where Mw is weight average molecular weight and Mn is number average molecular weight), polydisperse block length distribution, and/or polydisperse block number distribution, due, in an embodiment, to the effect of the shuttling agent(s) in combination with multiple catalysts used in their preparation.

In an embodiment, the ethylene/α-olefin multi-block copolymer can be produced in a continuous process and can have a polydispersity index (PDI defined as Mw/Mn) from 1.7 to 3.5, or from 1.8 to 3, or from 1.8 to 2.5, or from 1.8 to 2.2. When produced in a batch or semi-batch process, the ethylene/α-olefin multi-block copolymer can have Mw/Mn from 1.0 to 3.5, or from 1.3 to 3, or from 1.4 to 2.5, or from 1.4 to 2.

In addition, the ethylene/α-olefin multi-block copolymer can have a PDI (or Mw/Mn) fitting a Schultz-Flory distribution rather than a Poisson distribution. The present ethylene/α-olefin multi-block copolymer can have both a polydisperse block distribution as well as a polydisperse distribution of block sizes. This results in the formation of polymer products having improved and distinguishable physical properties. The theoretical benefits of a polydisperse block distribution have been previously modeled and discussed in Potemkin, Physical Review E (1998) 57 (6), pp. 6902-6912, and Dobrynin, J. Chem. Phys. (1997) 107 (21), pp 9234-9238.

The present ethylene/α-olefin multi-block copolymer can have a most probable distribution of block lengths.

The ethylene/α-olefin multi-block copolymer of the present disclosure, especially those made in a continuous, solution polymerization reactor, can have a most probable distribution of block lengths. For example, the ethylene multi-block copolymer can be defined as having:

(A) Mw/Mn from about 1.7 to about 3.5, at least one melting point, Tm, in degrees Celsius, and a density, d, in grams/cubic centimeter, where in the numerical values of Tm and d correspond to the relationship:

Tm>−2002.9+4538.5(d)−2422.2(d)², and/or

(B) Mw/Mn from about 1.7 to about 3.5, and is characterized by a heat of fusion, ΔH in J/g, and a delta quantity, ΔT, in degrees Celsius defined as the temperature difference between the tallest DSC peak and the tallest Crystallization Analysis Fractionation (“CRYSTAF”) peak, wherein the numerical values of ΔT and ΔH have the following relationships:

ΔT>−0.1299 ( )H)+62.81 for ΔH greater than zero and up to 130 J/g

ΔT>48° C. for ΔH greater than 130 J/g

wherein the CRYSTAF peak is determined using at least 5 percent of the cumulative polymer, and if less than 5 percent of the polymer has an identifiable CRYSTAF peak, then the CRYSTAF temperature is 30° C.; and/or

(C) elastic recovery, Re, in percent at 300 percent strain and 1 cycle measured with a compression-molded film of the ethylene/α-olefin interpolymer, and has a density, d, in grams/cubic centimeter, wherein the numerical values of Re and d satisfy the following relationship when ethylene/α-olefin interpolymer is substantially free of crosslinked phase:

Re>1481−1629(d); and/or

(D) has a molecular weight fraction which elutes between 40° C. and 130° C. when fractionated using temperature rising elution fractionation (TREF), characterized in that the fraction has a molar comonomer content of at least 5 percent higher than that of a comparable random ethylene interpolymer fraction eluting between the same temperatures, wherein said comparable random ethylene interpolymer has the same comonomer(s) and has a melt index, density and molar comonomer content (based on the whole polymer) within 10 percent of that of the ethylene/α-olefin interpolymer; and/or

(E) has a storage modulus at 25° C., G′(25° C.), and a storage modulus at 100° C., G′(100° C.), wherein the ratio of G′(25° C.) to G′(100° C.) is in the range of about 1:1 to about 9:1.

The ethylene/α-olefin multi-block copolymer can also have:

(F) molecular fraction which elutes between 40° C. and 130° C. when fractionated using TREF, characterized in that the fraction has a block index of at least 0.5 and up to about 1 and a molecular weight distribution, Mw/Mn, greater than about 1.3; and/or

(G) average block index greater than zero and up to about 1.0 and a molecular weight distribution, Mw/Mn greater than about 1.3.

It is understood that the ethylene/α-olefin multi-block interpolymer can have one, some, all, or any combination of properties (A)-(G). Block Index can be determined as described in detail in U.S. Pat. No. 7,608,668 herein incorporated by reference for that purpose. Analytical methods for determining properties (A) through (G) are disclosed in, for example, U.S. Pat. No. 7,608,668, Col. 31, line 26 through Col. 35, line 44, which is herein incorporated by reference for that purpose.

The ethylene/α-olefin multi-block interpolymer, and further copolymer, can comprise any one of properties (A) through (G), or may comprises a combination of two or more of (A) through (G).

Another type of ethylene/α-olefin multi-block interpolymers that can be used are those referred to as “mesophase separated”. The term “mesophase separation” means a process in which polymeric blocks are locally segregated to form ordered domains. Crystallization of the ethylene segments in these systems is primarily constrained to the resulting mesodomains and such systems may be referred to as “mesophase separated”. These mesodomains can take the form of spheres, cylinders, lamellae, or other morphologies known for block copolymers. The narrowest dimension of a domain, such as perpendicular to the plane of lamellae, is generally greater than about 40 nm in the mesophase separated block copolymers of the instant invention. The olefin block copolymer can be mesophase separated. Examples of these interpolymers may be found in, for example, International Publication Nos. WO/2009/097560, WO/2009/097565, WO/2009/097525, WO/2009/097529, WO/2009/097532, and WO/2009/097535, all of which are herein incorporated by reference.

With respect to the mesophase separated olefin block copolymers, the delta comonomer can be greater than 18.5 mol %, greater than 20 mol % or greater than 30 mol %. The delta comonomer can be from 18.5 mol % to 70 mol %, from 20 mol % to 60 mol % or from 30 mol % to 50 mol %. The term ‘delta comonomer’ means the difference in mole percent comonomer between the hard segment and the soft segment of the olefin block copolymer. The delta comonomer can be measured using ¹³C NMR such as described below and in U.S. Pat. No. 7,947,793. In certain embodiments, the ethylene/α-olefin multi-block interpolymer has a soft segment composed of from 20 mol % to 50 mol % derived from the comonomer (e.g., octene).

Monomers which can be used in preparing the present OBC include ethylene and one or more addition polymerizable monomers other than ethylene. Examples of addition polymerizable monomers other than ethylene include straight-chain or branched α-olefins of 3 to 30, preferably 3 to 20, more preferably 3 to 10 carbon atoms, such as propylene, 1-butene, 1-pentene, 3-methyl-1-butene, 1-hexene, 4-methyl-1-pentene, 3-methyl-1-pentene, 1-octene, 1-decene, 1-dodecene, 1-tetradecene, 1-hexadecene, 1-octadecene and 1-eicosene; cyclo-olefins of 3 to 30, preferably 3 to 20, carbon atoms, such as cyclopentene, cycloheptene, norbornene, 5-methyl-2-norbornene, tetracyclododecene, and 2-methyl-1,4,5,8-dimethano-1,2,3,4,4a,5,8,8a-octahydro-naphthalene; di- and polyolefins, such as butadiene, isoprene, 4-methyl-1,3-pentadiene, 1,3-pentadiene, 1,4-pentadiene, 1,5-hexadiene, 1,4-hexadiene, 1,3-hexadiene, 1,3-octadiene, 1,4-octadiene, 1,5-octadiene, 1,6-octadiene, 1,7-octadiene, ethylidenenorbornene, vinyl norbornene, dicyclopentadiene, 7-methyl-1,6-octadiene, 4-ethylidene-8-methyl-1,7-nonadiene, and 5,9-dimethyl-1,4,8-decatriene; and 3-phenylpropene, 4-phenylpropene, 1,2-difluoroethylene, tetrafluoroethylene, and 3,3,3-trifluoro-1-propene. More preferred α-olefins include propylene, 1-butene, 1-pentene, 1-hexene, 1-heptene and 1-octene, and more preferably include propylene, 1-butene, 1-hexene and 1-octene.

The olefin block copolymers can be produced via a chain shuttling process such as described in U.S. Pat. No. 7,858,706, which is herein incorporated by reference. In particular, suitable chain shuttling agents and related information are listed in Col. 16, line 39 through Col. 19, line 44. Suitable catalysts are described in Col. 19, line 45 through Col. 46, line 19 and suitable co-catalysts in Col. 46, line 20 through Col. 51 line 28. The process is described throughout the document, but particularly in Col. Col 51, line 29 through Col. 54, line 56. The process is also described, for example, in the following: U.S. Pat. No. 7,608,668; U.S. Pat. No. 7,893,166; and U.S. Pat. No. 7,947,793. Further exemplary catalytic processes include those disclosed in U.S. Pat. No. 8,785,554, which is herein incorporated by reference.

The ethylene/α-olefin multi-block interpolymer can have a density greater than 0.850 grams per cubic centimeter (g/cc), greater than 0.860 g/cc, or greater than 0.865 g/cc. The density can be up to 0.950 g/cc, up to 0.925 g/cc, up to 0.900 g/cc, or up to 0.890 g/cc. Density is measured by the procedure of ASTM D-792 or ISO 1183.

The ethylene/α-olefin multi-block interpolymer, can have a peak melting temperature of at least 110 or at least 115 and no more than 140, no more than 130, or no more than 125 ° C. The peak melting temperature is measured by differential scanning calorimetry (DSC) method described in U.S. Publication 2006/0199930 (WO 2005/090427), incorporated herein by reference.

The ethylene/α-olefin multi-block interpolymer can have a melt index (I2) from 0.1 grams per 10 minutes (g/10 min) or from 0.5 g/10 min to 50, to 40, to 30, or to 20 g/10 min as determined using ASTM D-1238 or ISO 1133 (190° C., 2.16 kg load).

The fibers can be monofilaments.

The resin used to make the fiber can comprise at least 70, at least 80, at least 90, at least 95, or at least 99 weight percent olefin block copolymer. The resin can consist essentially of or consist of olefin block copolymer.

Commercially available examples of such olefin block copolymers include INFUSE™ resins from The Dow Chemical Company.

### EXAMPLES

### Test Methods

### Melt Index

Melt index, or I2, is determined according to ASTM D1238 at 190° C., 2.16 kg for ethylene-based resins, and is reported in grams eluted per 10 minutes (g/10 mins).

### Melt Flow Rate

Melt flow rate, or MFR2, is determined according to ASTM D1238 at 230° C., 2.16 kg for propylene-based resins, and is reported in grams eluted per 10 minutes (g/10 mins).

### Density

Samples are prepared according to ASTM D1928. Measurements are made using ASTM D792, Method B, and are reported in grams per cubic centimeter (g/cc).

Differential Scanning calorimetry (DSC)

DSC is measured on a Mettler Toledo DSC 822e/700/Ro according to ISO 11357-1 Ed. 2013. The temperature range is from −100° C. to 250° C. with a temperature increase of 10° C./min. The peak melting point is determined from the 2nd heating cycle according to ISO 11357-1 Ed. 2013, and is reported in degrees Celsius (° C.).

### Solidification Point

The solidification and modulus development of the materials can be studied with a DHR-3 rheometer from TA Instruments equipped with an environmental temperature control system. The oven cavity is purged with N² (g) to avoid oxidation. The rheometer is preheated to 160° C. for at least 30 minutes prior to the measurements. The instrument is equipped with 8 mm diameter parallel plates and the initial sample gap is set to 1800 μm. Samples are compression-molded into 2 mm thick sheets at 150° C. using a Collin press. A disk of 8 mm diameter is punched out and placed on the bottom plate of the preheated rheometer. The sample is melted before the gap is closed and excess sample is trimmed off 300 seconds (s) temperature equilibration time are allowed before launching the measurement. Viscoelastic properties are measured at an angular frequency of 10 rad/s and a strain amplitude of 10% while the sample is cooled from 160° C. to 49° C. at a rate of −2° C./min. At the end of the ramp, the samples are held at 49° C. for an additional 300 s. To avoid loss of contact between sample and the top plate of the rheometer due to shrinkage during cooling, a zero axial force condition is imposed, which results in a gap reduction during the measurement.

The solidification point is defined as the temperature at which the viscosity of a resin reaches 40,000 Pa·s, and is reported in degrees Celsius (° C.).

### Denier

5 meters of a single filament is weighed, and then multiplied by 1,800 to obtain the denier (reported in grams/9,000 meters). This test is repeated a total of 5 times and the average is determined and reported in Table 4.

### Elastic Filament Hysteresis Test (Table 4)

A 2 cycle hysteresis test is performed according to ASTM D 5459 Ed. 2012 with a speed of 250 mm/min and a grip distance of 50 mm. The cycle elongation is 300%. The preload is set to 0.05 N and the test is performed with a single filament. Hysteresis loss 1st. cycle in [%]=(area under 1st. cycle elongation curve−area under 1st cycle unload curve)/area under 1st. cycle elongation curve). Hysteresis loss 2nd. cycle in [%]=(area under 2nd. cycle elongation curve−area under 2nd. cycle unload curve)/area under 2nd. cycle elongation curve).

### Elastic Filament Tensile Test (Table 4)

Tensile test is performed according to ISO 527-3 with a speed of 250 mm/min and a grip distance of 100 mm and preload of 0.05 N. The force at 200% elongation, the force at 400% elongation, and the elongation at break is measured and reported in Table 4.

### Elastic Laminate Hysteresis Test (Table 6)

A 2 cycle hysteresis test is performed according to ASTM D 5459 Ed. 2012 with a speed of 250 mm/min and a grip distance of 50 mm. The sample width is 50 mm. The cycle elongation is 100%.

Unload force at 50% of the second cycle is recorded and reported in N/50 mm.

Elastic laminate tensile test (Table 6)

Tensile test is performed according to ISO 527-3 with a speed of 250 mm/min and a grip distance of 100 mm and preload of 0.05 N. The sample width is 50 mm.

The force at 50% elongation, force at 100% elongation, maximum force, elongation at maximum force, and the elongation at 5N are recorded and reported in Table 6. Maximum force is the highest value of force achieved during tensile measurements according to ISO 527-3. The sample width is 50 mm.

### Example 1

Resin is fed into a 20 mm extruder. The temperature profile of the extruder is shown below in Table 1 (Zone 1-Zone 4). The resin is transformed in molten phase by heating and shearing and supplied to a 1 hole die via a spin pump and adapter. 1 mm, 2 mm, and 3 mm hole diameter dies are used during the experiments.

At the exit of the die, the molten resin is converted into a thick fiber, which is cooled in a water bath in a manner similar to as shown in FIG. 1. The water surface to die surface distance is 12 cm (air gap). The temperature of the water is 25° C. The filament is then cold stretched using four rollers to a particular stretch ratio, as summarized in Table 2. The stretch ratio is the velocity of the final (e.g. 4^{th}) roller divided by the velocity of the 1st roller. The tension is released and the filaments are collected by an air gun into a bag. Immediately after collection the filaments are wound by hand around a roll without tension.

The resin characteristics are set out in Table 3 and the testing results for the filaments were tested according to the above methods and the results are shown in Table 4. The inventive examples generally show the best balance of properties with similar or better hysteresis loss (lower is better), lower force at elongation (lower is better for comfort in diapers and incontinence products) and higher elongation at break (allowing for fewer sizes of products to be required).

TABLE 1
Process conditions

| | |
|---|---|
| Extruder Zone 1 [° C.] | 40 |
| Extruder Zone 2 [° C.] | 170 |
| Extruder Zone 3 [° C.] | 210 |
| Extruder Zone 4 [° C.] | 220 |
| Spin pump Temperature [° C.] | 220 |
| Adapter Temperature [° C.] | 230 |
| Die Temperature [° C.] | 230 |
| Throughput [gram/minute] | 15 |
| Air gap [cm] | 12 |
| Water Temperature [° C.] | 25 |
| Water path [cm] | 42 |
| Roll temperature 1-4 [° C.] | 70 |
| Final Take up velocity [m/min] | 350 |

TABLE 2
Stretch ratio (Cold Stretch)

| | Nominal | 1st roller | 2nd roller | 3rd roller | 4th roller |
|---|---|---|---|---|---|
| Stretch | elongation | velocity | velocity | velocity | velocity |
| ratio | [%] | [m/min] | [m/min] | [m/min] | [m/min] |
| 1.0 | 0 | 350 | 350 | 350 | 350 |
| 2.2 | 120 | 160 | 207 | 269 | 350 |
| 3.4 | 240 | 104 | 156 | 234 | 350 |
| 4.9 | 390 | 71 | 121 | 205 | 350 |

TABLE 3
Materials

| | | Melt Index (I2) | | | |
|---|---|---|---|---|---|
| | | [g/10] min or Melt | | | |
| | | Flow Rate | | DSC peak | |
| | | (MFR2) [g/10 | | melting temp | Solidification |
| Resin | Type | min]* | Density [g/cc] | [° C.] | point [° C.] |
| INFUSE ™ 9507 | Olefin block | 5.0 | 0.866 | 122 | 96 |
| available from | copolymer | | | | |
| The Dow | | | | | |
| Company | | | | | |
| (Midland, MI) | | | | | |
| ENGAGE ™ | Random | 5.0 | 0.870 | 65 | <50 |
| 8200, available | ethylene- | | | | |
| from The Dow | based | | | | |
| Chemical | elastomer | | | | |
| Company | | | | | |
| (Midland, MI) | | | | | |
| VERSIFY ™ | Random | 8.0 | 0.865 | 95 | 56 |
| 3401, available | propylene- | | | | |
| from The Dow | based | | | | |
| Chemical | elastomer | | | | |
| Company | | | | | |
| (Midland, MI) | | | | | |
| LYCRA | Thermoplastic | nm | nm | nm | nm |
| HyFit ® fiber | polyurethane | | | | |
| available from | (TPU) | | | | |
| INVISTA | | | | | |
| (Wichita, | | | | | |
| Kansas) | | | | | |

*For INFUSE ™ 9507 and ENGAGE ™ 82000 MI is tested and or VERSIFY ™ 3401 MFR is tested according to the ASTM1238 as stated under methodology.

TABLE 4
Elastic filament results

| | | Die | | | | Hysteresis | Hysteresis | Force at | Force at | Elongation |
|---|---|---|---|---|---|---|---|---|---|---|
| | | size | Stretch | Draw | Denier | loss 1^{st} | loss 2^{nd} | 200% | 400% | at break |
| Sample | Type | (mm) | ratio | ratio* | (g/9000 m) | cycle (%) | cycle (%) | (cN/den) | (cN/den) | (%) |
| Comparative | TPU | n/a | n/a | n/a | 770 | 61 | 37 | 0.117 | 0.403 | 500 |
| A | | | | | | | | | | |
| Comparative | ENGAGE ™ | 2 | 1.0 | 6.7 | 552 | nm | nm | nm | nm | 100 |
| B1 | 8200 | | | | | | | | | |
| Comparative | ENGAGE ™ | 2 | 2.2 | 5.5 | 803 | nm | nm | nm | nm | 156 |
| B2 | 8200 | | | | | | | | | |
| Comparative | ENGAGE ™ | 2 | 3.4 | 5.3 | 868 | nm | nm | nm | nm | 165 |
| B3 | 8200 | | | | | | | | | |
| Comparative | ENGAGE ™ | 2 | 4.9 | 5.3 | 883 | nm | nm | nm | nm | 158 |
| B4 | 8200 | | | | | | | | | |
| Comparative | VERSIFY ™ | 2 | 1.0 | 5.6 | 778 | 86 | 49 | 0.141 | 0.230 | 429 |
| C1 | 3401 | | | | | | | | | |
| Comparative | VERSIFY ™ | 2 | 2.2 | 4.5 | 1246 | 86 | 52 | 0.118 | 0.170 | 559 |
| C2 | 3401 | | | | | | | | | |
| Comparative | INFUSE ™ | 1 | 1.0 | 3.1 | 648 | 79 | 43 | 0.074 | 0.173 | 460 |
| D1 | 9507 | | | | | | | | | |
| Inventive D2 | INFUSE ™ | 1 | 2.2 | 2.6 | 931 | 55 | 34 | 0.040 | 0.090 | 670 |
| | 9507 | | | | | | | | | |
| Inventive D3 | INFUSE ™ | 1 | 3.4 | 2.3 | 1117 | 57 | 36 | 0.035 | 0.074 | 720 |
| | 9507 | | | | | | | | | |
| Inventive D4 | INFUSE ™ | 1 | 4.9 | 2.3 | 1121 | 57 | 34 | 0.039 | 0.076 | 690 |
| | 9507 | | | | | | | | | |
| Comparative | INFUSE ™ | 2 | 1.0 | 6.2 | 635 | 80 | 44 | 0.091 | 0.213 | 430 |
| E1 | 9507 | | | | | | | | | |
| Inventive E2 | INFUSE ™ | 2 | 2.2 | 5.1 | 958 | 62 | 37 | 0.042 | 0.092 | 660 |
| | 9507 | | | | | | | | | |
| Inventive E3 | INFUSE ™ | 2 | 3.4 | 5.0 | 985 | 54 | 33 | 0.041 | 0.075 | 860 |
| | 9507 | | | | | | | | | |
| Inventive E4 | INFUSE ™ | 2 | 4.9 | 4.7 | 1085 | 64 | 42 | 0.050 | 0.102 | 600 |
| | 9507 | | | | | | | | | |
| Comparative | INFUSE ™ | 3 | 1.0 | 9.3 | 640 | 73 | 38 | 0.136 | nm | 350 |
| F1 | 9507 | | | | | | | | | |
| Inventive F2 | INFUSE ™ | 3 | 2.2 | 7.6 | 957 | 64 | 33 | 0.061 | 0.140 | 500 |
| | 9507 | | | | | | | | | |
| Inventive F3 | INFUSE ™ | 3 | 3.4 | 7.1 | 1106 | 59 | 32 | 0.043 | 0.100 | 670 |
| | 9507 | | | | | | | | | |
| Inventive F4 | INFUSE ™ | 3 | 4.9 | 7.0 | 1113 | 66 | 41 | 0.045 | 0.097 | 690 |
| | 9507 | | | | | | | | | |

nm-filament breaks.
*Draw ratio is determined according to the following formula:
Draw ratio = (Diameter of a hole of the die)/(Average diameter of a filament)
Average diameter of a filament is calculated according to the following equation:
$Average diameter of a filament == \sqrt{\frac{( 4 * Denier )}{( 9000 * density * π )}} where Denier is expressed in kg \text{/} 9000 m , density in kg \text{/} {m}^{3} .$

### Example 2

Laminates are made substantially accord to the following process: 45 filaments with about 5 mm distance between the filaments are fixed to a metal frame with hooks. The filament length in unstretched state is 15 cm. Then the filaments are stretch to 60 cm (about 300%).

A predefined amount of adhesive is applied on the siliconized paper. The adhesive is spread across the paper with a roll and the weight of the siliconized paper and the adhesive is measured. The paper with the adhesive is applied on the filaments from the top side. The side of the paper with adhesive is kept on the filaments for about 30 seconds and then it is removed and the weight was measured again. The adhesive is a liquid low temperature glue and is applied at about 0.08 g/m.

The same approach is used from the bottom side of the filaments.

After that two sheets of a non-woven material (where the fibers are a bicomponent spunbond PP core/ASPUN™ 6000 sheath and the non-woven has a structure having SMMMS structure (spun bond outer layers of non-woven with three melt blown inner layers of non-woven, 15 gsm) is applied from the bottom and the top side of the filaments. The load of ˜1 kg per 21×29 cm2 is applied for approximately 30 seconds. After that the load is removed and the filaments are allowed to retract back to original size. The laminates are tested at least 24 h after the production.

The laminates were tested as set out above and the test results are shown in Table 5. The inventive laminates show a desired lower force at extension, show higher extension at 5N force, and show a desired lower unload force as compared to laminates made with TPO fibers.

TABLE 5
Laminate results

| | | Total | Force | Force | | | | Unload |
|---|---|---|---|---|---|---|---|---|
| | | Weight | at | at | Max | | Elongation | Force at |
| | Filament | of | 50% | 100% | Force | Elongation | at | 50% 2 |
| | Denier | filaments | [N/50 | [N/50 | [N/50 | at Max | 5N/50 mm | cycles |
| Sample | (g/9000 m) | [g] | mm] | mm] | mm] | Force [%] | [%] | [N/50 mm] |
| Comparative | 760 | 0.6 | 2.5 | 4.7 | 73 | 380 | 105 | 1.7 |
| 2 (Uses | | | | | | | | |
| Comparative | | | | | | | | |
| TPU) | | | | | | | | |
| Inventive 2.1 | 960 | 0.8 | 1.4 | 2.3 | 55 | 360 | 160 | 0.8 |
| (uses fibers | | | | | | | | |
| Inventive F2) | | | | | | | | |
| Inventive 2.2 | 1100 | 0.9 | 1.65 | 2.6 | 61 | 390 | 170 | 0.9 |
| (uses fibers | | | | | | | | |
| inventive F3) | | | | | | | | |

This disclosure further encompasses the following aspects.

Aspect 1: A method of manufacturing an article (preferably an elastic article) comprising: providing a resin (preferably having a peak melting temperature) comprising an olefin block copolymer; spinning the resin into one or more fibers; cooling the one or more fibers to below the solidification point of the resin; after cooling, stretching the one or more fibers to a nominal elongation in the range of 50% to 900% to form one or more stretched fibers; and relaxing the one or more stretched fibers (preferably to form an elastic fiber).

Aspect 2: The method of aspect 1, wherein the nominal elongation is in the range of 100% to 400%.

Aspect 3: The method of any one of the preceding aspects, wherein the olefin block copolymer is characterized by one or more of the following: a density in the range of 0.850 to 0.950 g/cc, preferably 0.860 to 0.890 g/cc (preferably according to ASTM D-792); a melt index (I2) in the range of 0.1 to 50, preferably 0.5 to 30 g/10 minutes (preferably according to ASTM 1238 at 190° C. and 2.16 kg load); and a peak melting temperature in the range of 110 to 140, preferably 115° C. to 125° C. (preferably according to ISO-11357).

Aspect 4: The method of any one of the preceding aspects, wherein the stretching occurs over two or more rollers (preferably including a first roller and a final roller) and the one or more fibers are stretched to a stretch ratio of 1.5 to 10.

Aspect 5: The method of aspect 4 wherein the two or more rollers are heated to a temperature in the range of 60° C. to 80° C.

Aspect 6: The method of any one of the preceding aspects, wherein the spinning step comprises: heating the resin to a temperature above a peak melting temperature of the resin to form a molten resin; extruding the molten resin through a die comprising one or more holes having diameter(s) in the range of 0.2 mm to 6 mm; and after extrusion, drawing the molten resin into one or more fibers at a draw ratio in the range of 3 to 30.

Aspect 7: The method of any one of the preceding aspects, wherein the fiber consists essentially of the olefin block copolymer.

Aspect 8: The method of any one of the preceding aspect wherein the fiber consists of the olefin block copolymer and other optional polymers or optional additives in amounts of less than 10, preferably less than 5, more preferably less than 1 weight percent based on total weight of the fiber.

Aspect 9: The method of any one of the preceding aspects wherein the one or more fibers are monofilaments.

Aspect 10: The method of any one of the preceding aspects wherein the olefin block copolymer is not cross-linked.

Aspect 11: The method of any one of the preceding aspects further comprising, after stretching, laminating the one or more fibers to one or more flexible substrates.

Aspect 12: The method of aspect 11 wherein the laminating occurs before relaxing the one or more stretched fibers.

Aspect 13: The method of aspect 11 wherein the laminating occurs while the fibers are stretched.

Aspect 14: The method of any one of claims 10 to 13 wherein the laminating comprises laminating the one or more stretched fibers between two flexible substrates.

Aspect 15: The method of any one of claims 10 to 14 wherein the one or more flexible substrates comprise a non-woven substrate.

Aspect 16: An elastic article made from the method of any one of claims 1-15.

Aspect 17 An elastic article comprising one or more elastic fibers bonded to a nonwoven, wherein the one or more elastic fibers comprises an olefin block copolymer and wherein the article has one or more of the following properties: a force at 50% elongation of less than 2 Newtons; a force at 100% elongation of less than 4 Newtons; an elongation at five Newtons for a 50 mm sample of the laminate of at least 120%; an unload force at 50% on a second cycle of less than 1.5 N/50 mm.

Aspect 18: A diaper or incontinence product comprising the article of claim 16 or 17.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). Moreover, stated upper and lower limits can be combined to form ranges (e.g. “at least 1 or at least 2 weight percent” and “up to 10 or 5 weight percent” can be combined as the ranges “1 to 10 weight percent”, or “1 to 5 weight percent” or “2 to 10 weight percent” or “2 to 5 weight percent”).

Unless specified to the contrary herein, all test standards are the most recent standard in effect as of the filing date of this application, or, if priority is claimed, the filing date of the earliest priority application in which the test standard appears.

1. A method of manufacturing an article comprising:
providing a resin comprising an olefin block copolymer;
spinning the resin into one or more fibers;
cooling the one or more fibers to below the solidification point of the resin;
after cooling, stretching the one or more fibers to a nominal elongation in the range of 50% to 900% to form one or more stretched fibers; and
relaxing the one or more stretched fibers.

2. The method of claim 1, wherein the nominal elongation is in the range of 100% to 400%.

3. The method of claim 1, wherein the olefin block copolymer is characterized by one or more of the following:
a density in the range of 0.850 to 0.950 g/cc, preferably 0.860 to 0.890 g/cc according to ASTM D-792;
a melt index (12) in the range of 0.1 to 50, preferably 0.5 to 30 g/10 minutes according to ASTM 1238 at 190° C. and 2.16 kg load; and
a peak melting temperature in the range of 110 to 140, preferably 115° C. to 125° C. according to ISO-11357.

4. The method of claim 1, wherein the stretching occurs over two or more rollers and the one or more fibers are stretched to a stretch ratio of 1.5 to 10.

5. The method of claim 4 wherein the two or more rollers are heated to a temperature in the range of 60° C. to 80° C.

6. The method of claim 1, wherein the spinning step comprises:
heating the resin to a temperature above a peak melting temperature of the resin to form a molten resin;
extruding the molten resin through a die comprising one or more holes having diameter(s) in the range of 0.2 mm to 6 mm; and
after extrusion, drawing the molten resin into one or more fibers at a draw ratio in the range of 3 to 30.

7. The method of claim 1, wherein the fiber consists essentially of the olefin block copolymer.

8. The method of claim 1, wherein the one or more fibers are monofilaments.

9. The method of claim 1 wherein the olefin block copolymer is not cross-linked.

10. The method of claim 1 further comprising, after stretching, laminating the one or more fibers to one or more flexible substrates.

11. The method of claim 10 wherein the laminating occurs before relaxing the one or more stretched fibers.

12. The method of claim 9 wherein the laminating comprises laminating the one or more stretched fibers between two flexible substrates.

13. An elastic article made from the method of claim 1.

14. An elastic article comprising one or more elastic fibers bonded to a nonwoven, wherein the one or more elastic fibers comprises an olefin block copolymer and wherein the article has one or more of the following properties: a force at 50% elongation of less than 2 Newtons; a force at 100% elongation of less than 4 Newtons; an elongation at five Newtons for a 50 mm sample of the laminate of at least 120%; an unload force at 50% on a second cycle of less than 1.5 N/50 mm.

15. A diaper or incontinence product comprising the article of claim 13.
